(19) United States
(12) Patent Application Publication
(10) Pub. No.: US 2016/0174875 A1
(43) Pub. Date: Jun. 23, 2016

(54) Assembly for the Extraction of Respiratory Gas Samples

(71) Applicants: SIEMENS AKTIENGESELLSCHAFT, München (DE); SIEMENS HEALTHCARE DIAGNOSTICS HOLDING GMBH, Eschborn (DE)

(72) Inventors: Brigitte FÖRSTER, München (DE); Andreas KAPPEL, Glashuetten (DE); Erhard MAGORI, Feldkirchen (DE); Roland POHLE, Ottehhofen (DE); Oliver von SICARD, München (DE)

(73) Assignees: Siemens Aktiengesellschaft, München (DE); Siemens Healthcare Diagnostics Holding GmbH, Eschborn (DE)

(21) Appl. No.: 14/910,042

(22) PCT Filed: Aug. 6, 2014
(86) PCT No.: PCT/EP2014/066883

§ 371 Date: Feb. 4, 2016

(30) Foreign Application Priority Data
Aug. 8, 2013 (DE) 10 2013 215 640.5

(51) Int. Cl. A61B 5/097; A61M 39/22

(52) U.S. Cl. CPC A61B 5/097; A61M 39/22

(57) ABSTRACT

An assembly for the extraction of respiratory gas samples may include a container for receiving a respiratory gas sample, a piston arranged in the container in a movable and gas-sealing manner, and a gas feed into the container, which gas feed can be connected to a mouthpiece.

### CROSS-REFERENCE TO RELATED APPLICATIONS

This application is a U.S. National Stage Application of International Application No. PCT/EP2014/066883 filed Aug. 6, 2014, which designates the United States of America, and claims priority to DE Application No. 10 2013 215 640.5 filed Aug. 8, 2013, the contents of which are hereby incorporated by reference in their entirety.

### TECHNICAL FIELD

The invention relates to an arrangement for taking respiratory gas samples.

### BACKGROUND

There has previously been no reliable and at the same time economical method of diagnosing diseases such as, for example, tuberculosis. The measurement of marker gases characteristic of particular diseases in human exhalation air represents a noninvasive technique with high potential for also being economically usable in order to detect diseases such as tuberculosis and metabolic disorders. The analysis of exhalation air may, for example, be carried out by gas chromatography/mass spectrometry (GC/MS). To this end, the exhalation air needs to be collected beforehand and stored. The storage has been carried out in the prior art with so-called adsorption tubes. This storage works reliably even over weeks, which for example allows dispatch worldwide.

In order to collect enough molecules in an adsorption tube, a certain amount of respiratory air (about 1 l) needs to be conveyed through the adsorption tube. In this case, it is necessary to comply continuously with a particular flow rate (for instance 100 ml/min), so that the gas spends a sufficiently long residence time in the adsorption tube and the molecules can adhere to the adsorbent. These constraints cannot be satisfied for direct sampling from exhalation air since on the one hand the tubes have a high flow resistance so that it is not possible to exhale directly through the tubes, and on the other hand taking of the breath sample lasts about 10 minutes. In order to solve this problem, plastic bags are used as breath storage units. These in turn have the disadvantage that they release gases which significantly vitiate the sample of the exhalation air.

### SUMMARY

One embodiment provides an arrangement for taking respiratory gas samples, the arrangement comprising a container for receiving a breath sample, a piston arranged so as to be displaceable and slide in a gas-sealing fashion in the container, a gas delivery into the container, which can be connected to a mouthpiece, a reception device for receiving an adsorption tube, and means for conveying the breath sample out of the container to the adsorption tube.

In a further embodiment, the volume formed by the container and the piston is between 0.1 l and 3 l, e.g., between 0.5 l and 1.5 l.

In a further embodiment, the container internally has a PTFE surface.

In a further embodiment, the piston has a PTFE surface on the outer faces that touch the container.

In a further embodiment, the container has a glass or metal surface on the inner faces that face toward the breath sample.

In a further embodiment, the piston has a glass or metal surface on the face that faces toward the breath sample.

In a further embodiment, the arrangement includes a pump for controlled delivery of the breath sample out of the container.

In a further embodiment, the arrangement includes a valve system configured in order to discharge a first fraction of the delivered exhalation gas into the surroundings and to convey a second part, following the first, of the delivered exhalation gas into the container.

In a further embodiment, the arrangement includes a flow sensor.

In a further embodiment, the arrangement includes a throttle for generating a backpressure of 15 mm water column.

In a further embodiment, the flow sensor comprises pressure sensors for determining the difference between the pressures before and after the throttle.

In a further embodiment, the arrangement includes a heating device.

In a further embodiment, the arrangement includes a sensor for determining the distance traveled by the piston.

In a further embodiment, the arrangement includes a pressure sensor in the container.

### BRIEF DESCRIPTION OF THE DRAWINGS

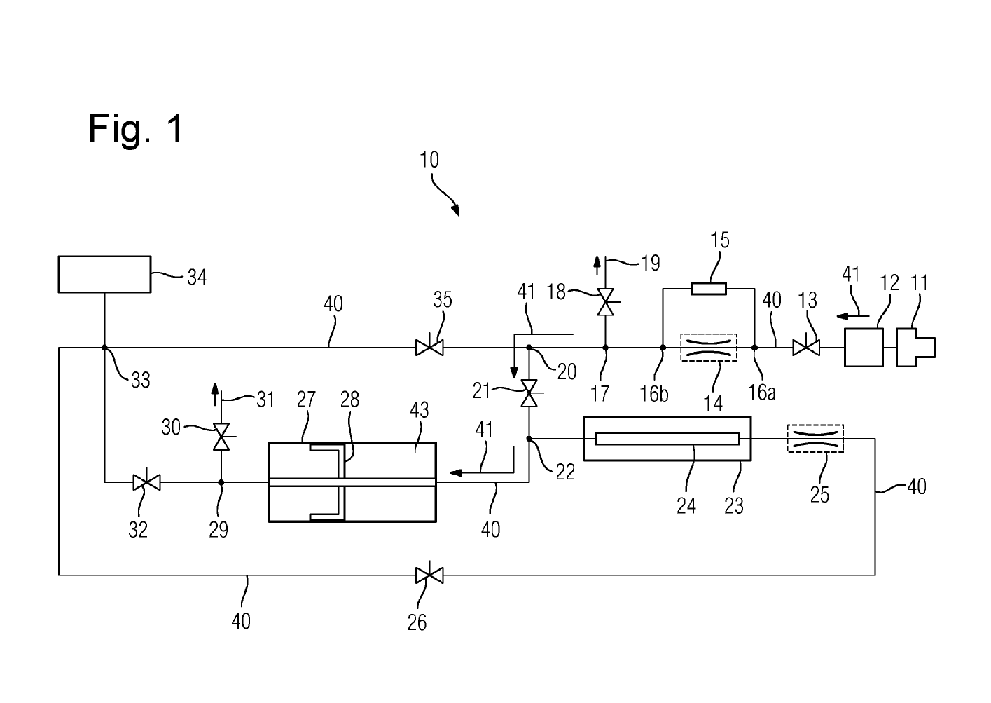

An example embodiment of the invention is explained in detail below with reference to FIG. 1, which shows an example sampling system for respiratory gas samples.

### DETAILED DESCRIPTION

Embodiments of the present invention provide an improved arrangement for taking respiratory gas samples

In one embodiment, the arrangement for taking respiratory gas samples comprises a container for receiving a respiratory gas sample, a piston arranged so as to be displaceable and slide in a gas-sealing fashion in the container, and a gas delivery into the container, which can be connected to a mouthpiece. The container may, for example, be a cylinder.

The container and the piston together form a storage volume for a respiratory gas sample, which can be increased by retracting the piston from the container. The maximum storage volume is preferably between 0.1 l and 3 l, in particular between 0.5 l and 1.5 l.

In this case the container and the piston are preferably configured in such a way that the surfaces facing toward the respiratory gas sample, i.e. the inner surfaces, comprise materials which cause no, or essentially no, degassing, for example PTFE (polytetrafluoroethylene), glass or metal. The advantageous effect achieved by this is that a respiratory gas sample stored in the container is not, or not significantly, contaminated. To this end, it is possible for the container and/or the piston to have a coating of the corresponding material. The container and/or the piston may also be made entirely, or substantially entirely, of the material.

The arrangement provided in this way can be flushed well between different filling processes so that no residues remain in the system. In other words, the container is reusable. For example, this avoids the need to replace a storage bag when taking a respiratory gas sample, so that the outlay is reduced.

In one embodiment, the container and/or the piston have a PTFE surface on the faces which touch the other respective element. In this way, particularly low-friction displacement of the piston in the container is made possible. At the same time, degassing is furthermore also avoided. Low-friction displacement is particularly advantageous in order to facilitate filling of the container with the respiratory gas sample by human exhalation pressure, the piston having to be displaced during filling by the exhalation pressure.

Expediently, the arrangement comprises a reception device for receiving an adsorption tube, and means for conveying the respiratory gas sample out of the container to the adsorption tube. The means are in this case expediently one or more valves. This makes it possible to convey the respiratory gas sample temporarily stored in the container into a replaceable adsorption tube in a controlled way. Advantageously, to this end the arrangement comprises a pump for controlled delivery of the respiratory gas sample out of the container.

In one embodiment, the arrangement comprises a valve system, which is configured in order to discharge a first fraction of the delivered exhalation gas into the surroundings and to convey a second part, following the first, of the delivered exhalation gas into the container. The effect achieved by this is that the components of the delivered exhalation gas, which come for example from the mouth, pharynx and trachea and which could vitiate the breath sample, are not delivered, or are delivered only in a small quantity, to the container. A typical first part of the exhalation gas to be discarded comprises between 0.25 l and 0.75 l, ideally 0.5 l.

In this case, the breath is initially fed into a bypass. The volume of the breath blown in is measured with a flow sensor (integral over the flow). After a desired volume is reached, the valves switch the breath flow from the bypass into the storage piston. An arrangement in which the breath storage unit is closed with a prestressed passive valve may be envisioned. Initially, the breath is fed into a “bypass breath storage unit” with the desired bypass volume. When this bypass breath storage volume is full, the pressure in the system increases and the passive valve opens to the main breath storage unit. The main breath storage unit is therefore not filled until a desired volume has flowed into the bypass breath storage unit. This solution saves on the volume-controlled switchover when blowing in.

The arrangement may comprise a flow sensor. With the flow sensor, for example, it is possible to check the speed of the influx of the exhalation gas, and monitoring of the filling of the container can therefore be carried out. If the arrangement comprises controlling electronics, for example, a measurement value is therefore available for the control.

The arrangement may comprise a throttle for generating a backpressure in the region of about 150 Pa (corresponding to 15 mm H₂O). A backpressure in this range advantageously closes the velum (soft palate) and therefore prevents or reduces the entry of perturbing respiratory gas components from the paranasal sinuses. In this case, it is expedient to balance the backpressure of the throttle with the backpressure existing anyway in the arrangement, for example because of the displacement of the piston, in order overall to maintain a backpressure which is as low as possible.

If a throttle is provided, the flow sensor may advantageously be provided by pressure sensors before and after the throttle. The pressure sensors may determine the pressure difference between their respective positions and therefore allow calculated deduction of the flow rate with the aid of the properties of the throttle. In this case, the pressure sensors may specifically be configured in order to determine the absolute pressure two times, the pressure difference then being calculated. Likewise, one or both of the pressure sensors may be configured in order to determine the pressure difference directly.

The arrangement may comprise a heating device. By thermal regulation of the container, piston and/or line system, adsorption of gas components in the heated regions is reduced or avoided. The respiratory gas sample therefore has its gas composition preserved better, and contaminations by gas residues of previous respiratory gas samples are reduced.

The arrangement may furthermore comprise a sensor for determining the distance traveled by the piston. In this way, monitoring and control of the filling of the container is possible. As an alternative or in addition, a pressure sensor may be provided in the container. This likewise allows control of the filling.

FIG. 1 shows a sampling system 10 for respiratory gas samples.

The sampling system 10 comprises a mouthpiece 11, by way of which subject can deliver a respiratory gas sample, i.e. breathe out into the sampling system 10. The sampling system 10 per se in this case only comprises a reception device for the mouthpiece 11, the mouthpiece 11 itself being a replaceable element. The mouthpiece 11 is connected to a system of gas lines 40, which connect the further elements of the sampling system 10 to one another and make it possible to forward and distribute the respiratory gas sample and other gases. The mouthpiece 11 is followed by a bacteria filter 12, by which bacteria are removed from the respiratory gas sample. The bacteria filter 12 is also expediently replaceable.

The bacteria filter 12 is followed in an influx direction 41, which a respiratory gas sample essentially follows, by a first valve 13. This is further followed by a first node point 16a, a throttle 14 with a diameter of 0.3 mm and a second node point 16b. The first and second node points 16a, b are configured for the connection of a flow meter 15. For example, pressure sensors, which are in turn interconnected in such a way that a pressure difference between the two node points 16a, b is output, may be arranged at the two node points 16a, b. A control device (not represented in FIG. 1), determines the flow rate through the throttle 14 from the pressure difference.

The second node point 16b is followed in the influx direction 41 by a third node point 17, from which a gas outlet 19 can be reached via a second valve 18. In the influx direction 41, the third node point 17 is followed by a fourth node point 20, a third valve 21 and a fifth node point 22. Connected directly to the fifth node point 22, there is a cylinder 27 in which a displaceable piston 28 is arranged on an axis. On the influx side, on which the gas line 40 opens into the cylinder 27 in the influx direction 41, the cylinder 27 with the piston 28 forms a respiratory gas temporary storage volume 43.

On the side of the cylinder 27 facing away from the influx, the gas line is continued to a sixth node point 29, which leads via a fourth valve 30 to a second gas outlet 31. The sixth node point 29 is furthermore connected via a fifth valve 32 to a seventh node point 33. Between the seventh node point 33 and the fourth node point 20, there is a further connection via a sixth valve 35. Lastly, another gas line 40 leads from the fifth node point 22 to a device 23 for receiving an adsorption tube 24. After the adsorption tube 24, the gas line 40 continues via a second throttle 25 with a diameter of 0.1 mm to a seventh valve 26, and from there to the seventh node point 33.

Lastly, the seventh node point 33 is connected to a pump 34. In this example, the cylinder 27 is made of stainless steel, the inner face being coated with PTFE. The piston is in turn made of PTFE. In this way, low friction is ensured during displacement, and at the same time it is ensured that no degassing from the piston 28 or the cylinder 27 causes contamination of the respiratory gas sample.

For the same reason, it is expedient for the further elements, insofar as is possible, to consist of PTFE, glass or metal. For example, the elements of the valves 13, 18, 21, 26, 30, 32, 35 which are in contact with the gas may consist of stainless steel and Teflon tubes may be used as gas lines 40.

In order to take a respiratory gas sample the following steps are carried out.

First, all components of the sampling system 10 are flushed in order to remove residues of possibly preceding samples. To this end, the valves 13, 18, 21, 26, 30, 32, 35 are suitably driven and from the outside air is sucked through the sampling system 10 by means of the pump 34.

The piston 28 is pushed in the cylinder 27 for further preparation into a position in which the respiratory gas temporary storage volume 43 is minimized as far as possible. Lastly, a mouthpiece 11 for the subject is fitted onto the reception device for the mouthpiece 11 and an adsorption tube 24 is inserted into the device 23.

It will be assumed below that a subject exhales/blows forcefully into the mouthpiece 11. A control device for the sampling system 10 initially switches the valves 13, 18, 21, 26, 30, 32, 35 in such a way that the first fourth of a liter of the respiratory gas sample, which comes from the mouth/pharynx, does not enter the cylinder. To this end, the first and second valves 13, 18 are opened and the third and sixth valves 21, 35 are closed. By means of the flow sensor 15, the amount of respiratory gas that flows through the throttle 14, and is therefore currently discarded, can be monitored. Once the first fourth of a liter of the respiratory gas sample has flowed through the throttle 14, the valves are switched over in order to convey the respiratory gas along the influx direction 41 into the cylinder 27. To this end, the first and third valves 13, 21 are opened and the second, sixth and seventh valves 18, 35, 26 are closed.

By the exhalation pressure exerted by the subject, the piston 28 is displaced in the cylinder 27 in order to make space for the exhalation air in the respiratory gas temporary storage volume 43. The respiratory gas sample is therefore stored in the respiratory gas temporary storage volume 43. After an establishable amount of air has flowed into the cylinder 27, for example 1 liter in addition to the initially discarded fourth of a liter, the control device ends the collection of respiratory gas by closing at least the first valve 13.

Subsequently, the respiratory gas sample is fed through the adsorption tube 24 in such a way that the best possible adsorption of contained gases takes place. To this end the third, sixth and fifth valves 21, 35, 32 are closed and the seventh and fourth valves 26, 30 are opened. The pump ensures a corresponding pressure buildup, which draws the respiratory gas sample from the respiratory gas temporary storage volume 43 through the adsorption tube 24. The second throttle 25 in this case in turn ensures sufficiently high flow resistance, i.e. a sufficiently low flow rate, which allows good adsorption of the gases in the adsorption tube 24.

The adsorption tube may then be removed and is available, for example, for a GC/MS analysis in order to determine the concentration of marker gases. With the first described step, the sampling system 10 can be prepared for a further subject.

In order to avoid condensation in the sampling system 10, the apparatus may be heated to a temperature above the dew point of respiratory air. As an alternative, a desiccant, for example silica gel, may also be used in the mouthpiece. The desiccant must not, however, adsorb any relevant marker gases. As an alternative, condensation of respiratory air may be tolerated. The accumulating condensate may be removed by enough flushing processes. As an alternative or in addition, drainage devices may be provided in the sampling system 10.

For subsequent evaluation, it is advantageous for an adsorption tube 24 which preferably adsorbs hydrocarbons and reduces the adsorption of water to be used as the adsorption tube 24, in order to reduce the high proportion of water and the associated influence on the measurement during the subsequent evaluation of the gas constituents.

In order to facilitate the process control, a pressure sensor may be provided in the region of the respiratory gas temporary storage volume 43. The pressure sensor registers, for example, a pressure rise when the possibility of the piston 28 to move in the cylinder 27 is exhausted, i.e. the cylinder 27 is fully filled with respiratory gas. The control device may thereupon end the sampling. Likewise, the pumping dry of the cylinder 27 may be monitored.

What is claimed is:

1. An arrangement for taking respiratory gas samples, the arrangement comprising:
a container configured to receive a breath sample,
a piston arranged to slide in the container in a gas-sealing manner,
a first gas delivery system providing a gas flow into the container, and configured for connection to a mouthpiece,
a reception device configured to receive an adsorption tube, and
a second gas delivery system configured to communicate the breath sample from the container to the adsorption tube.

2. The arrangement of claim 1, wherein the container and the piston define a volume between 0.1 liter and 3 liter.

3. The arrangement of claim 1, wherein an internal surface of the container comprises PTFE.

4. The arrangement of claim 1, wherein the piston has a PTFE surface on outer faces that touch the container.

5. The arrangement of claim 1, wherein the container has a glass or metal surface on an inner faces that face toward the breath sample.

6. The arrangement of claim 1, wherein the piston has a glass or metal surface on a face that faces toward the breath sample.

7. The arrangement of claim 1, comprising a pump configured to provide controlled delivery of the breath sample out of the container.

8. The arrangement of claim 1, comprising a valve system configured to discharge a first fraction of the delivered exhalation gas into a surrounding area and to convey a second part of the delivered exhalation gas, following the first part, into the container.

9. The arrangement of claim 1, comprising a flow sensor.

10. The arrangement of claim 1, comprising a throttle configured to generate a backpressure of 15 mm water column.

11. The arrangement of claim 9, wherein the flow sensor comprises pressure sensors configured to determine a difference between a pressure upstream of the throttle and a pressure downstream of the throttle.

12. The arrangement of claim 1, comprising a heating device.

13. The arrangement of claim 1, comprising a sensor configured to determine a distance traveled by the piston.

14. The arrangement of claim 1, comprising a pressure sensor in the container.
